(19) United States
(12) Patent Application Publication
(10) Pub. No.: US 2018/0049859 A1
(43) Pub. Date: Feb. 22, 2018

(54) INTRAVASCULAR FLOW DIVERSION DEVICES

(71) Applicant: Spartan Micro, Inc., Round Rock, TX (US)

(72) Inventors: Eric P. STOPPENHAGEN, Round Rock, TX (US); Mark DIAS, Round Rock, TX (US)

(21) Appl. No.: 15/480,223

(22) Filed: Apr. 5, 2017

Related U.S. Application Data
(60) Provisional application No. 62/375,595, filed on Aug. 16, 2016.

(51) Int. Cl. A61F 2/01

(52) U.S. Cl. CPC A61F 2/01; A61F 2002/016; A61F 2002/011

(57) ABSTRACT

Described herein are devices and methods for closing abnormal communications between one or more vessels or a vessel and an organ. The devices and methods described herein are configured to divert a flow of a body fluid such as, for example, blood or bile away from the abnormal conduit thereby promoting thrombosis and occlusion in the case of blood vessel to blood vessel communication or an aneurysm and/or decrease in size of and epithelization of the communication such as in the case of a fistula.

### CROSS-REFERENCE

This application claims the benefit of U.S. Provisional Application No. 62/375,595, filed Aug. 16, 2016, which application is incorporated herein by reference.

### BACKGROUND

An aneurysm is an area of enlargement of a portion of a wall of an artery that results from a weakening in the wall of the artery. Some aneurysms have a saccular shape with a neck that is continuous with the arterial wall and that opens into the artery so that blood flows from the artery through the neck and into the interior of the aneurysm.

Typically, an aneurysm has a risk of enlarging and/or rupturing over time due to continued flow of blood through the aneurysm (i.e. because the blood flow is at relatively high pressure and the wall of the aneurysm is weak).

Traditional treatment modalities for aneurysms include surgical treatment and treatment through intravascular delivery of implants to the aneurysm. In general, intravascular implants used to treat intracranial aneurysms are designed to prevent blood flow from traveling from the blood vessel into the aneurysm.

Fistulas are abnormal connections between a hollow vessel or organ and another hollow vessel or organ. Examples of fistulas include arteriovenous fistulas and biliary fistulas. When there is flow of body fluid from one organ or vessel through the fistula it tends to keep the fistula patent. Treatment of fistulas traditionally involves the prevention of flow of a body fluid through the fistula. Fistulas are usually treated through reducing or diverting flow through the fistula or surgical ligation.

### SUMMARY

Described herein are devices and methods for closing abnormal communications between one or more vessels or a vessel and an organ. The devices and methods described herein are configured to divert a flow of a body fluid such as, for example, blood or bile away from the abnormal conduit thereby promoting thrombosis and occlusion in the case of blood vessel to blood vessel communication or an aneurysm and/or decrease in size of and epithelization of the communication such as in the case of a fistula.

The traditional treatment of intracranial aneurysms includes surgical craniotomy with direct placement of clips on the outside of the aneurysm in order to close or seal the aneurysm. Sealing the aneurysm with clips via the craniotomy procedure is done in order to prevent flow of blood into the aneurysm. Craniotomy is highly invasive with a relatively high associated morbidity and long recovery time. The traditional treatment of aneurysms also includes intravascular techniques that are relatively less invasive than surgical procedures involving craniotomy. In one such traditional technique, an embolic coil is deployed within an aneurysm. The method of deployment of an embolic coil is based on the coils ability to block and/or promote clot formation within the interior of the aneurysm so that the interior of the aneurysm is filed and thus blood is prevented from flowing into it. This method for aneurysm occlusions is dependent on the ability to densely pack an aneurysm with multiple coils, however the recurrence of the treated aneurysm can occur over time if the coil compacts and allows blood to recirculate within the aneurysm. Additionally, not all aneurysms are treatable with coils, for example, wide necked aneurysms are not able to be coiled as the coil mass could migrate out of the aneurysm.

The devices and methods described herein offer benefits over the traditional treatment modalities in that, for example, the devices and methods described herein are employed intravascularly thus are less invasive than craniotomy procedures. As compared to traditional intravascular treatments, the devices and methods described herein require delivery of a single device only, typically, and the device is configured to conform to the local anatomy thus anchoring the device in place and preventing device migration.

The devices and methods described herein are configured for delivery to the site of treatment via a catheter under fluoroscopic guidance. The device, in some embodiments, comprises components, that when the device is deployed, conform to the local anatomy at the deployment site and work to restrict flow into an abnormality, such as a cerebral aneurysm or a cardio vascular condition such as patent ductus arterosus. Restriction of flow into the abnormal conduit or passage causes a thrombogenic biological response and secondarily a growth of endothelial cells. The thrombogenic response is mechanically initiated from the implantation of the device and works to hinder the flow of blood into the abnormality. Over time, the growth of endothelial cells over the implanted device scaffolding acts as a biological seal to fully occlude the abnormality.

In some embodiments of the methods described herein, a flow diverting implant as described herein is delivered to and deployed at least partially within an aneurysm so that a portion of the implant is within the saccular interior of the aneurysm, a portion of the implant is within the neck of the aneurysm and a portion of the implant is within the blood vessel near the opening of the neck to the blood vessel. In some embodiments of the flow diverting implant described herein, one or more elements of the implant are shape conforming elements configured to conform to a shape of either the aneurysm interior, the neck of the aneurysm, or the blood vessel near the opening of the neck to the blood vessel.

The flow diversion device described herein is configured to restrict flow through the components of the device triggering thrombosis and eventual occlusion of the abnormal conduit or vascular abnormality such as an aneurysm.

In some embodiments of the flow diversion implant described herein, the flow diversion implant comprises two spiral shaped components. The first spiral shaped component acts as a conforming anchor to be deployed into the abnormality. The proximal end of the first spiral is attached to an expanding tubular mesh which complies to the neck of the abnormality and is attached on the opposite end to the second spiral shaped component which conforms to the adjacent wall in the parent vessel.

Materials suitable for manufacturing components of the devices described herein include shape memory alloys such as, for example, nickel-titanium which when deployed from a linear delivery geometry, for example, when compressed within a delivery catheter, will return to an expanded geometry (e.g. a flat spiral or circle). The elements of the device are generally constructed in a fashion to maximize surface area contact with the internal vasculature, have dimensions which allow the elements to maintain internal surface wall apposition within the vasculature and hinder blood flow into the abnormality for the purpose of diverting flow from the abnormality and restoring blood flow through the parent vessel. Refer to FIG. 2 for some general dimensions of the spiral component. Other materials suitable for use in manufacturing the devices described herein include precious metals such as platinum, gold, tantalum, and silver which are radiodense materials and are beneficial for vision under fluoroscopic guidance.

In some embodiments of the flow diverting implant described herein, at least a portion of the flow diverting implant is coated with a thin film polymer to further hinder blood flow into the abnormality. The surface of one or more coated components may also be treated with agents that induce and promote thrombosis and/or endothelial cell growth which would hasten complete occlusion of the abnormality.

Described herein is a flow diverting implant for treating an aneurysm, the aneurysm having an interior, a neck, and receiving a blood flow from a blood vessel having a blood vessel wall that opens into the neck, the flow diverting implant comprising: a first shape conforming element and a second shape conforming element each having a delivery configuration and a deployment configuration; a mesh positioned between the first shape conforming element and the second shape conforming element; wherein the delivery configuration comprises a compressed configuration and the deployment configuration comprises an expanded configuration; and wherein when in the deployment configuration, the first shape conforming element is configured to conform to the interior, the mesh is configured to conform to the neck, and the second shape conforming element is configured to conform to the blood vessel wall so that the flow diverting implant is anchored in place by the first shape conforming element and the second shape conforming element and diverts the blood flow away from the aneurysm. In some embodiments of the flow diverting implant, the first shape conforming element and the second shape conforming element each comprise a coil. In some embodiments of the flow diverting implant, when in the delivery configuration the coil is substantially linearly shaped and in the deployment configuration the coil is substantially circularly shaped. In some embodiments of the flow diverting implant, when in the deployment configuration the coil is substantially coiled within a single plane. In some embodiments of the flow diverting implant, the first shape conforming element and the second shape conforming element are configured so that they must be compressed in order to be substantially linearly shaped. In some embodiments of the flow diverting implant, the first shape conforming element and the second shape conforming element comprises a memory metal. In some embodiments of the flow diverting implant, the memory metal causes the first shape conforming element and the second shape conforming element to be substantially circularly shaped when the first shape conforming element and the second shape conforming element are not compressed. In some embodiments of the flow diverting implant, the first shape conforming element and the second shape conforming element are compressed when inside a lumen of a delivery catheter. In some embodiments of the flow diverting implant, the implant comprises a radiopaque metal. In some embodiments of the flow diverting implant, the implant comprises a polymer coating configured to block blood flow through the flow diverting implant when in the deployment configuration.

Described herein is a method for treating an aneurysm with a flow diverting implant comprising a first shape conforming element and a second shape conforming element separated by a mesh, the method comprising: delivering the flow reducing implant to the aneurysm; expanding the first shape conforming element within an interior of the aneurysm; positioning the mesh within a neck of the aneurysm; expanding the second shape conforming element within a blood vessel that opens into the neck of the aneurysm; wherein the first shape conforming element conforms to the interior of the aneurysm and the second shape conforming element conforms to the blood vessel thereby anchoring the flow diverting implant and diverting a blood flow to the aneurysm. In some embodiments of the method, the first shape conforming element and the second shape conforming element each comprise a coil. In some embodiments of the method, when in the delivery configuration the coil is substantially linearly shaped and in the deployment configuration the coil is substantially circularly shaped. In some embodiments of the method, when in the deployment configuration the coil is substantially coiled within a single plane. In some embodiments of the method, the first shape conforming element and the second shape conforming element are configured so that they must be compressed in order to be substantially linearly shaped. In some embodiments of the method, the first shape conforming element and the second shape conforming element comprises a memory metal. In some embodiments of the method, the memory metal causes the first shape conforming element and the second shape conforming element to be substantially circularly shaped when the first shape conforming element and the second shape conforming element are not compressed. In some embodiments of the method, the first shape conforming element and the second shape conforming element are compressed when inside a lumen of a delivery catheter. In some embodiments of the method, the flow reducing implant comprises a radiopaque metal. In some embodiments of the method, the flow reducing implant comprises a polymer coating configured to block blood flow through the flow diverting implant when in the deployment configuration.

Described herein is a flow diverting implant for treating an aneurysm, the aneurysm having an interior, a neck, and receiving a blood flow from a blood vessel having a blood vessel wall that opens into the neck, the flow diverting implant comprising: a first shape conforming element and a second shape conforming element each having a delivery configuration and a deployment configuration; an elastic middle element connecting the first shape conforming element to the second shape conforming element; wherein the delivery configuration comprises a compressed configuration and the deployment configuration comprises an expanded configuration; wherein when in the deployment configuration, the first shape conforming element is configured to conform to the interior, the elastic middle element is positioned within the neck, and the second shape conforming element is configured to conform to the blood vessel wall; and wherein when in the deployment configuration, the elastic middle element is configured to generate an elastic force that is applied to the first shape conforming element and the second shape conforming element thereby securing the first shape conforming element against a surface of the interior and securing the second shape conforming element against a surface of the blood vessel so that the flow diverting implant is anchored in place by the first shape conforming element and the second shape conforming element and diverts the blood flow away from the aneurysm. In some embodiments, the first shape conforming element and the second shape conforming element each comprise a coil. In some embodiments, when in the delivery configuration the coil is substantially linearly shaped and in the deployment configuration the coil is substantially circularly shaped. In some embodiments, when in the deployment configuration the coil is substantially coiled within a single plane. In some embodiments, the first shape conforming element and the second shape conforming element are configured so that they must be compressed in order to be substantially linearly shaped. In some embodiments, the first shape conforming element and the second shape conforming element comprises a memory metal. In some embodiments, the memory metal causes the first shape conforming element and the second shape conforming element to be substantially circularly shaped when the first shape conforming element and the second shape conforming element are not compressed. In some embodiments, the first shape conforming element and the second shape conforming element are compressed when inside a lumen of a delivery catheter. In some embodiments, the flow diverting implant comprises a radiopaque metal. In some embodiments, the flow diverting implant comprises a polymer coating configured to block blood flow through the flow diverting implant when in the deployment configuration.

Described herein is method for treating an aneurysm with a flow diverting implant comprising a first shape conforming element and a second shape conforming element separated by an elastic middle element, the method comprising: (a) delivering the flow reducing implant to the aneurysm; (b) expanding the first shape conforming element within an interior of the aneurysm; (c) positioning the elastic middle element within a neck of the aneurysm; (d) expanding the second shape conforming element within a blood vessel that opens into the neck of the aneurysm; wherein the elastic middle element generates an elastic force that secures the first shape conforming element against a surface of the interior of the aneurysm and the second shape conforming element against a surface of the blood vessel thereby anchoring the flow diverting implant in place so that it diverts blood flow away from the aneurysm. In some embodiments, the first shape conforming element and the second shape conforming element each comprise a coil. In some embodiments, when in the delivery configuration the coil is substantially linearly shaped and in the deployment configuration the coil is substantially circularly shaped. In some embodiments, when in the deployment configuration the coil is substantially coiled within a single plane. In some embodiments, the first shape conforming element and the second shape conforming element are configured so that they must be compressed in order to be substantially linearly shaped. In some embodiments, the first shape conforming element and the second shape conforming element comprises a memory metal. In some embodiments, the memory metal causes the first shape conforming element and the second shape conforming element to be substantially circularly shaped when the first shape conforming element and the second shape conforming element are not compressed. In some embodiments, the first shape conforming element and the second shape conforming element are compressed when inside a lumen of a delivery catheter. In some embodiments, the flow reducing implant comprises a radiopaque metal. In some embodiments, the flow reducing implant comprises a polymer coating configured to block blood flow through the flow diverting implant when in the deployment configuration.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an illustration of the anatomical path of travel of a catheter containing a flow diverting implant as described herein.

FIG. 2A shows an embodiment of a flow diverting implant in a deployment configuration.

FIG. 2B shows a top view of an embodiment of a shape conforming element in a deployment configuration, wherein the shape conforming element comprises a spiral geometry having one or more turns.

FIG. 2C shows a lateral view of a flow diverting implant.

FIG. 2D shows an illustration of an embodiment of a flow diverting implant in a deployment configuration within an aneurysm.

FIG. 3 shows an illustration of an exemplary method for using a flow diverting implant as described herein to occlude an abnormal conduit in the body of a patient.

### DETAILED DESCRIPTION

Described herein are devices and methods for occluding an abnormal conduit within the body of a patient. Non-limiting examples of abnormal conduits within the body of a patient that are treated with the devices and methods described herein include intracranial aneurysms, abdominal aortic aneurysms, thoracic aneurysms, patent ductus arteriosus, patent foramen ovale, arteriovenous fistula, gastric fistula, and biliary fistula.

In general, the devices described herein comprise flow diversion implants configured to be implanted within or in proximity to an abnormal conduit within the body of a patient and further configured to cause a diversion of flow of a body fluid away from the conduit. With the diversion of flow of the body fluid, an occluding clot forms within the conduit and/or the conduit is occluded via epithelialization. In the treatment of aneurysms, occlusion of the aneurysm neck (i.e. an abnormal conduit) leads to occlusion of the aneurysm which prevents additional flow of blood to the aneurysm and thus prevents rupture of the aneurysm.

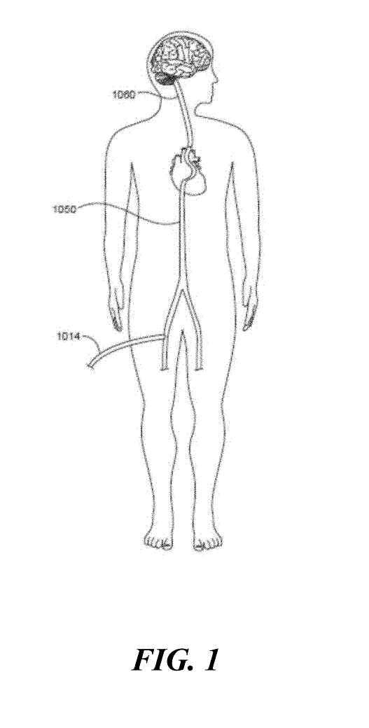

Embodiments of a flow diverting implant as described herein are configured for delivery to a target location via a traditional catheter or micro-catheter that is configured to deploy the implant at the target location. For example, FIG. 1 shows an illustration of the anatomic path of travel of a catheter 1014 containing a flow diverting implant as described herein. The catheter 1014 may be inserted into a femoral artery of a patient (using, for example, Seldinger technique), advanced up through the aorta 1050 of the patient, and from there the catheter 1014 may be advanced up through a carotid artery 1060 to an intracranial target location such as an intracranial aneurysm where a flow diverting implant may be deployed in order to, for example, occlude the aneurysm thus preventing aneurysm rupture.

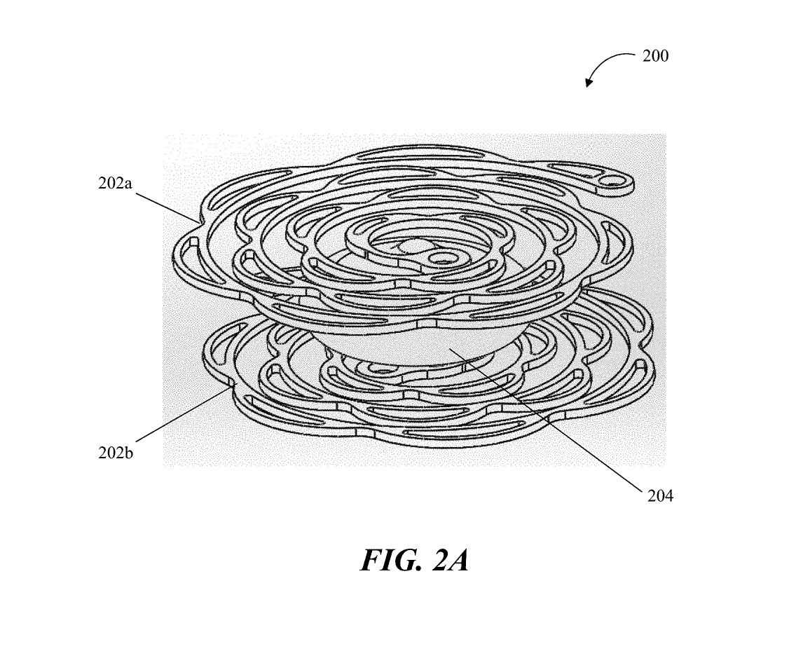

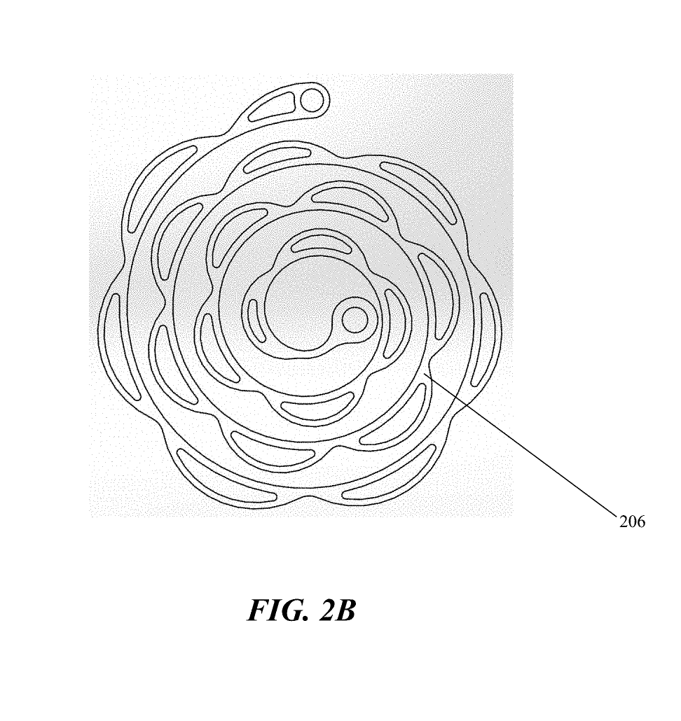

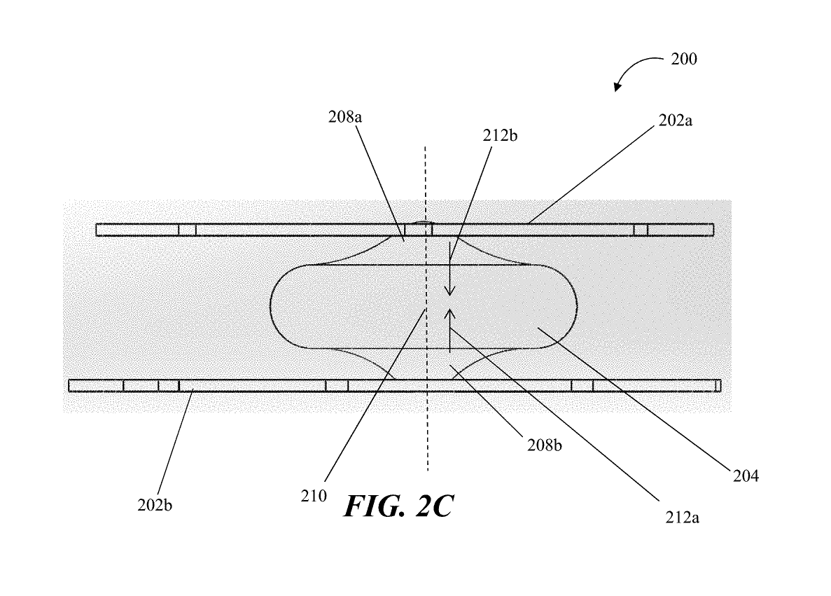

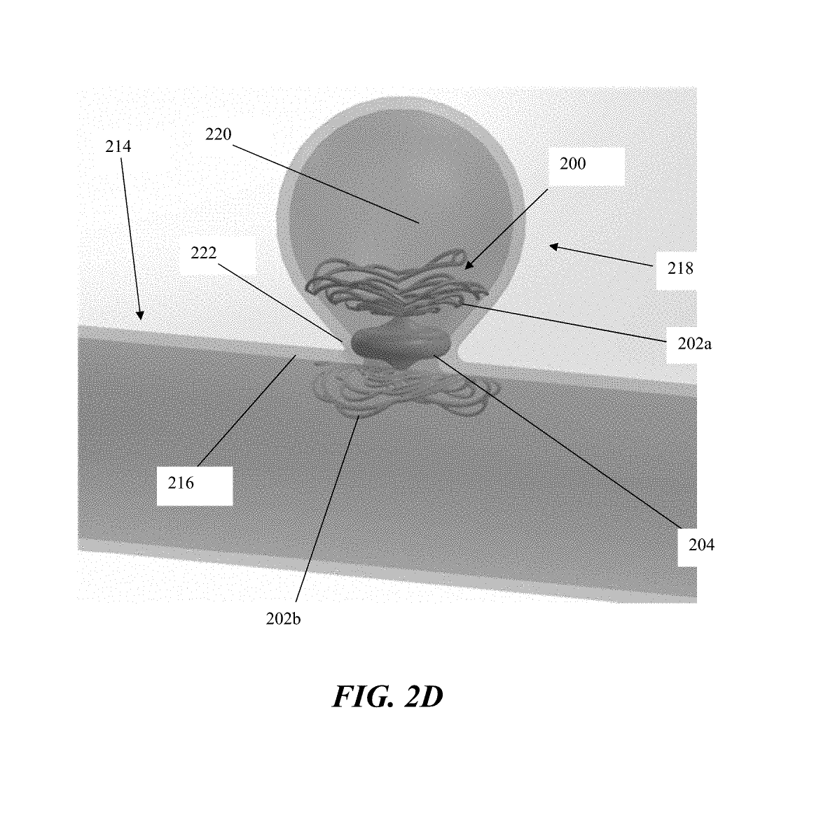

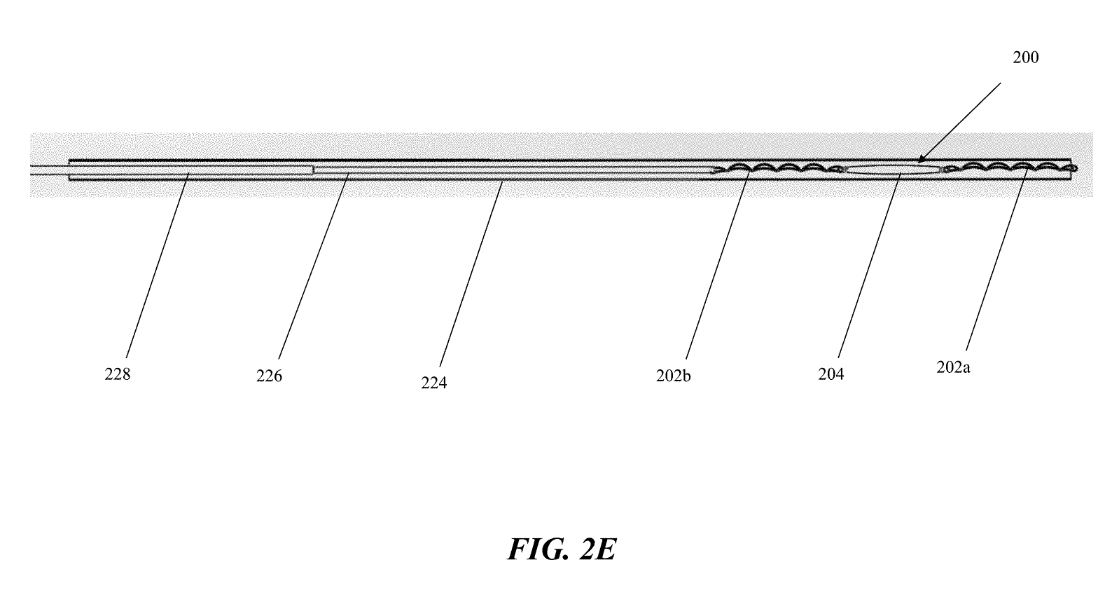

FIG. 2A shows an embodiment of a flow diverting implant 200 in a deployment configuration. As shown, a flow diverting implant 200 comprises a first shape conforming element 202a, a second shape conforming element 202b, and a middle element 204. The flow diverting implant as described herein has a deployment configuration (as shown) and a delivery configuration.

First and second shape conforming elements 202a and 202b, in some embodiments, are configured to have shape conforming properties so that they conform to a shape of a structure when deployed within or around that structure. For example, when the flow diverting implant 200 is deployed within an aneurysm, the first shape conforming element 202a is configured to expand to a deployment configuration which conforms to the shape of at least a portion of the interior of the aneurysm or aneurysm wall, and the second shape conforming element 202b is configured to expand to a deployment configuration which conforms to the shape of at least a portion of the wall of the blood vessel that opens into the aneurysm. In these embodiments, first shape conforming element 202a and second shape conforming element 202b are shape conforming in that they are made from relatively soft and compressible metals or alloys of such metals, and they have a geometry that provides shape conforming properties.

In general, when the flow diverting implant 200 is in a deployment configuration, the first shape conforming element 202a and the second shape conforming element 202b fit flush or substantially flush against the respective walls on each end of an abnormal conduit thus anchoring the flow diverting implant into position. In some embodiments of the flow diverting implant 200, the first and the second shape conforming elements 202a and 202b have a spiral geometry when the flow diverting implant 200 is in the deployment configuration.

The first and the second shape conforming elements 202a and 202b are each connected to a middle element 204, as shown in FIG. 2A. In some embodiments of the flow diverting implant 200, the middle element 204 comprises a compressible material such as, for example, a tubular mesh, or a polymer, or a hydrogel. In some embodiments of the flow diverting implant 200, the middle element 204 comprises an elastic material configured to generate an elastic force. In some embodiments of the flow diverting implant 200, the middle element 204 comprises a material configured to generate a tensile force, such as, for example, a spring. In some embodiments of the flow diverting implant 200, the middle element 204 comprises a material that is one or more of compressible, elastic, and configured to generate a tensile force.

In general, the middle element 204 is configured to be positioned within an abnormal conduit, such as, for example, a neck of an aneurysm and is anchored into place by the first and the second shape conforming elements 202a and 202b. In some embodiments of the flow diverting implant 200, when the middle element 204 is positioned within an abnormal conduit in the deployment configuration of the flow diverting implant 200, the middle element 204 fills the conduit and thus blocks flow of a body fluid through the conduit. For example, when the flow diverting implant 200 is deployed within an aneurysm the middle element 204 comprising, for example, a tubular mesh is positioned within the neck of the aneurysm and fills the interior of the neck of the aneurysm so that no blood flows from the blood vessel that opens into the neck to the aneurysm. In other words, the middle element 204 is configured to occlude an abnormal conduit within the body of a patient when deployed within that conduit.

In some embodiments of the flow diverting implant 200, when the implant 200 is in the deployment configuration, the middle element 204 causes the first and second shape conforming elements 202a and 202b to travel (or be pulled) towards each other thus shortening the length of the deployed diverting implant 200 and respectively pressing the first and second compressible elements 202a and 202b tightly against a surface or structure with which they are in contact. For example, in embodiments wherein the middle element 204 is elastic, when stretched through a neck of an aneurysm in a deployment configuration of the flow diverting implant 200, it will generate a tensile force that will draw the first and second compressible elements 202a and 202b towards each other.

FIG. 2B shows a top view of an embodiment of a shape conforming element in a deployment configuration, wherein the shape conforming element comprises a spiral geometry having one or more turns 206. As shown in FIGS. 2A-2C, in some embodiments, the spiral geometry of a shape conforming element is substantially flat in the deployment configuration so that each turn of the spiral 206 lies essentially within the same plane. In some embodiments, individual turns of the spiral 206 of each of the first and second shape conforming elements is configured to move out of the substantially flat geometry to allow for a flush fit against a non-flat surface. For example, when the flow diverting implant 200 is deployed within an aneurysm having an interior wall around the abnormal conduit to the aneurysm (i.e. the aneurysm neck) that has a convex shape, one or more turns of the spiral adjust so that the spiral geometry of the fist shape conforming element 202a matches the convex surface of the interior wall of the aneurysm forming a flush fit of the first shape conforming element 202a to the interior wall of the aneurysm.

FIG. 2C shows a lateral view of a flow diverting implant 200. Middle element 204 has a circular or annular shape that tapers on each end to form coupling points 208a and 208b where the middle element 204 is respectively coupled to the first and second shape conforming elements 202a and 202b. Middle element 204 has a longitudinal axis 210 along which, in some embodiments of the flow diverting implant 200, the middle element generates a compressive force in the deployed configuration that respectively draws the first and second shape conforming elements 202a and 202b towards each other along the direction illustrated by arrows 212a and 212b. In some of these embodiments of the flow diverting implant 200, the middle element 204 generates a compressive force via a spring within the middle element 204 along its longitudinal axis 210. In some of these embodiments of the flow diverting implant 200, the middle element 204 generates a compressive force when the middle element expands laterally outwards within the conduit shortening along its longitudinal axis 210. In these embodiments of the flow diverting implant 200, by compressing along its longitudinal axis 210 while in the deployment configuration (i.e. while positioned within an abnormal conduit) the middle element 204 pulls the first and second shape conforming elements 202a and 202b flush against the respective walls that are contiguous with the openings of the abnormal conduit. The pulling of the first and second shape conforming elements 202a and 202b along the direction of arrows 20a and 208b pulls them flush against the two sides of the abnormal conduit, thereby anchoring the flow diverting implant into position at the location of the abnormal conduit (e.g. an aneurysm).

FIG. 2D shows an illustration of an embodiment of a flow diverting implant 200 in a deployment configuration within an aneurysm 218. The aneurysm 218 is a saccular type aneurysm that expands out from wall 216 of the blood vessel 214. As shown, in the deployment configuration of the flow diverting implant 200, the flow diverting implant 200 is positioned to block blood flow from the blood vessel 214 in the aneurysm 218. Aneurysm 218 further comprises an interior 220 and a neck 222. As shown in FIG. 2D, the neck 222 of the aneurysm 218 is an abnormal conduit between blood vessel 214 and the aneurysm 218. Occluding of the neck 222 of the aneurysm 218 will lead to blood stasis within the interior of the aneurysm 220 that will cause clot formation there. Over time, the neck of the aneurysm 222 will epithelialize and thus permanently seal the neck 222 of the aneurysm 218. Here, in this example, the abnormal conduit that is ultimately sealed by the flow diverting implant 200 is the neck 222 of the aneurysm 218, but it should be understood that the same mechanism of occlusion occurs in other types of abnormal conduits within the body of a patient described here.

As shown in FIG. 2D, a flow diverting implant 200 comprises first and second shape conforming elements 202a and 202b connected to a middle element 204. In the deployment configuration of the flow diverting implant 200, a first shape conforming element 202a is positioned within an interior 220 of an aneurysm 218. The first shape conforming element 202a is configured to expand within the interior of the aneurysm 220 when the flow diverting implant 200 is in a deployment configuration, and the first shape conforming element 202a is further configured to conform to the wall of the aneurysm is that is around the opening of the neck 222 to the aneurysm so that it fits flush or essentially flush against the wall of the aneurysm 218. The second shape conforming element 202b is configured to expand within the blood vessel 214 when the flow diverting implant 200 is in a deployment configuration, and the second shape conforming element 202b is further configured to conform to the wall of the blood vessel 216 that is around the opening of the neck 222 to the blood vessel 216 so that it fits flush or essentially flush against the wall of the blood vessel 216. The middle element 204, in some embodiments of the flow diverting implant 200, expands within the neck 222 in order to occlude the neck 222 when the flow diverting implant is within the deployment configuration. In some embodiments, the middle element 204 generates an elastic force when positioned within the abnormal conduit 222 that draws the first and second shape conforming elements 202a and 202b towards each other so that they are flush against the openings of the abnormal conduit 222 (i.e. the aneurysm neck 222).

Materials suitable for manufacturing components of the devices described herein include shape memory alloys such as, for example, nickel-titanium. Other materials suitable for use in manufacturing the devices described herein include precious metals such as platinum, gold, tantalum, and silver which are radiodense materials and are beneficial for vision under fluoroscopic guidance.

In some embodiments of the flow diverting implant 200 one or more components of the flow diverting implant are coated with a coating that promotes occlusion of the aneurysm. For example, in some embodiments, the flow reducing implant 200 is partially or completely coated with a polymer that acts to prevent blood flow through the implant body. For example, in some embodiments, the flow reducing implant 200 is partially or completely coated with a thrombogenic material or substance in order to promote clot formation, for example, within an aneurysm.

FIG. 2D shows an embodiment of a flow diverting implant 200 in a delivery configuration. As shown in FIG. 2D, in a delivery configuration, the flow diverting implant 200 is compressed within a delivery conduit such as, for example, a micro-catheter 224. Micro-catheter 224 is configured to hold flow diverting implant 200 in a compressed configuration so that first and second shape conforming elements 202a and 202b have a linear or substantially linear configuration and middle element 204 is compressed into a linear or substantially linear shape as well. In some embodiments, a detachment tube 226 is detachably coupled to the flow diverting implant 200. In the embodiment of the detachment tube 226 shown in FIG. 2D, the detachment tube 226 is detachably coupled with the second shape conforming element 202b. In some embodiments, the detachment tube is caused to detach from the flow diverting implant 200 by a hand-held detachment device or grip tube 228. In some embodiments, withdrawing the detachment device or grip tube 228 in a proximal direction causes the detachment of the detachment tube 226 from the flow diverting implant 200 and when the micro-catheter 226 is withdrawn proximally, the flow diverting implant 200 self deploys thereby changing from the delivery configuration shown in FIG. 2D to the deployment configuration shown in FIGS. 2A-2C.

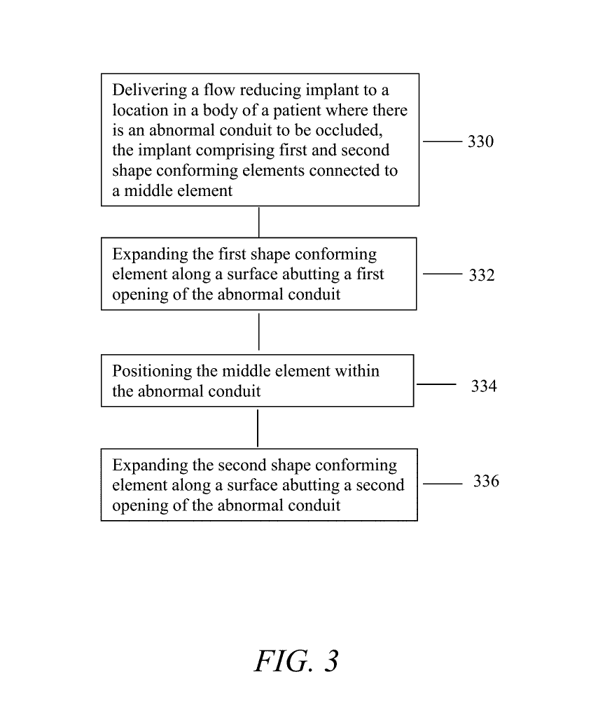

FIG. 3 shows an illustration of an exemplary method for using a flow diverting implant as described herein to occlude an abnormal conduit in the body of a patient. In a step 330, a flow diverting implant as described herein is delivered, for example, intravascularly inside the lumen of a micro-catheter, to an abnormal conduit within the body of a patient such as, for example, a neck of an aneurysm that connects a blood vessel to an interior of an aneurysm. While in the lumen of the catheter, the flow diverting implant is in a delivery configuration and is compressed. At the location of the abnormal conduit, the implant is deployed by, for example, withdrawing the catheter proximally and advancing the implant through the abnormal conduit so that the implant changes from a delivery configuration to a deployment configuration as described in the following steps which do not necessarily occur sequentially: In a step 332, the first shape conforming element expands against a surface on a first side of the abnormal conduit (e.g. the wall of an aneurysm). In a step 334, the middle element comprising, for example, a mesh, expands within the abnormal conduit (e.g. an aneurysm neck). In a step 336 the second shape conforming element expands against a surface on a second side of the abnormal conduit (e.g. the wall of a blood vessel that opens into the aneurysm). In some embodiments of the flow diverting implant described herein, the flow diverting implant is self-expanding.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A flow diverting implant for treating an aneurysm, the aneurysm having an interior, a neck, and receiving a blood flow from a blood vessel having a blood vessel wall that opens into the neck, the flow diverting implant comprising:
a first shape conforming element and a second shape conforming element each having a delivery configuration and a deployment configuration;
a mesh positioned between the first shape conforming element and the second shape conforming element;
wherein the delivery configuration comprises a compressed configuration and the deployment configuration comprises an expanded configuration; and
wherein when in the deployment configuration, the first shape conforming element is configured to conform to the interior, the mesh is configured to conform to the neck, and the second shape conforming element is configured to conform to the blood vessel wall so that the flow diverting implant is anchored in place by the first shape conforming element and the second shape conforming element and diverts the blood flow away from the aneurysm.

2. The flow diverting implant of claim 1, wherein the first shape conforming element and the second shape conforming element each comprise a coil.

3. The flow diverting implant of claim 2, wherein when in the delivery configuration the coil is substantially linearly shaped and in the deployment configuration the coil is substantially circularly shaped.

4. The flow diverting implant of claim 3, wherein when in the deployment configuration the coil is substantially coiled within a single plane.

5. The flow diverting implant of claim 4, wherein the first shape conforming element and the second shape conforming element are configured so that they must be compressed in order to be substantially linearly shaped.

6. The flow diverting implant of claim 5, wherein the first shape conforming element and the second shape conforming element comprises a memory metal, wherein the memory metal causes the first shape conforming element and the second shape conforming element to be substantially circularly shaped when the first shape conforming element and the second shape conforming element are not compressed, and wherein the first shape conforming element and the second shape conforming element are compressed when inside a lumen of a delivery catheter.

7. The flow diverting implant of claim 1, comprising a radiopaque metal.

8. The flow diverting implant of claim 1, comprising a polymer coating configured to block blood flow through the flow diverting implant when in the deployment configuration.

9. A method for treating an aneurysm with a flow diverting implant comprising a first shape conforming element and a second shape conforming element separated by a mesh, the method comprising:
(a) delivering the flow reducing implant to the aneurysm;
(b) expanding the first shape conforming element within an interior of the aneurysm;
(c) positioning the mesh within a neck of the aneurysm;
(d) expanding the second shape conforming element within a blood vessel that opens into the neck of the aneurysm;
wherein the first shape conforming element conforms to the interior of the aneurysm and the second shape conforming element conforms to the blood vessel thereby anchoring the flow diverting implant and diverting a blood flow to the aneurysm.

10. The method of claim 9, wherein the first shape conforming element and the second shape conforming element each comprise a coil.

11. The method of claim 10, wherein when in the delivery configuration the coil is substantially linearly shaped and in the deployment configuration the coil is substantially circularly shaped.

12. The method of claim 11, wherein when in the deployment configuration the coil is substantially coiled within a single plane.

13. The method of claim 12, wherein the first shape conforming element and the second shape conforming element are configured so that they must be compressed in order to be substantially linearly shaped.

14. The method of claim 13, wherein the first shape conforming element and the second shape conforming element comprises a memory metal, wherein the memory metal causes the first shape conforming element and the second shape conforming element to be substantially circularly shaped when the first shape conforming element and the second shape conforming element are not compressed, and wherein the first shape conforming element and the second shape conforming element are compressed when inside a lumen of a delivery catheter.

15. The method of claim 9, the flow reducing implant comprises a radiopaque metal.

16. The method of claim 9, the flow reducing implant comprises a polymer coating configured to block blood flow through the flow diverting implant when in the deployment configuration.

17. A flow diverting implant for treating an aneurysm, the aneurysm having an interior, a neck, and receiving a blood flow from a blood vessel having a blood vessel wall that opens into the neck, the flow diverting implant comprising:
a first shape conforming element and a second shape conforming element each having a delivery configuration and a deployment configuration;
an elastic middle element connecting the first shape conforming element to the second shape conforming element;
wherein the delivery configuration comprises a compressed configuration and the deployment configuration comprises an expanded configuration;
wherein when in the deployment configuration, the first shape conforming element is configured to conform to the interior, the elastic middle element is positioned within the neck, and the second shape conforming element is configured to conform to the blood vessel wall; and
wherein when in the deployment configuration, the elastic middle element is configured to generate an elastic force that is applied to the first shape conforming element and the second shape conforming element thereby securing the first shape conforming element against a surface of the interior and securing the second shape conforming element against a surface of the blood vessel so that the flow diverting implant is anchored in place by the first shape conforming element and the second shape conforming element and diverts the blood flow away from the aneurysm.

18. The flow diverting implant of claim 17, wherein the first shape conforming element and the second shape conforming element each comprise a coil, and wherein when in the delivery configuration the coil is substantially linearly shaped and in the deployment configuration the coil is substantially circularly shaped.

19. The flow diverting implant of claim 18, wherein when in the deployment configuration the coil is substantially coiled within a single plane.

20. The flow diverting implant of claim 19, wherein the first shape conforming element and the second shape conforming element are configured so that they must be compressed in order to be substantially linearly shaped.

21. The flow diverting implant of claim 20, wherein the first shape conforming element and the second shape conforming element comprises a memory metal, wherein the memory metal causes the first shape conforming element and the second shape conforming element to be substantially circularly shaped when the first shape conforming element and the second shape conforming element are not compressed, and wherein the first shape conforming element and the second shape conforming element are compressed when inside a lumen of a delivery catheter.

22. The flow diverting implant of claim 17, comprising a radiopaque metal.

23. The flow diverting implant of claim 17, comprising a polymer coating configured to block blood flow through the flow diverting implant when in the deployment configuration.

24. A method for treating an aneurysm with a flow diverting implant comprising a first shape conforming element and a second shape conforming element separated by an elastic middle element, the method comprising:
(a) delivering the flow reducing implant to the aneurysm;
(b) expanding the first shape conforming element within an interior of the aneurysm;
(c) positioning the elastic middle element within a neck of the aneurysm;
(d) expanding the second shape conforming element within a blood vessel that opens into the neck of the aneurysm;
wherein the elastic middle element generates an elastic force that secures the first shape conforming element against a surface of the interior of the aneurysm and the second shape conforming element against a surface of the blood vessel thereby anchoring the flow diverting implant in place so that it diverts blood flow away from the aneurysm.

25. The method of claim 24, wherein the first shape conforming element and the second shape conforming element each comprise a coil, and wherein when in the delivery configuration the coil is substantially linearly shaped and in the deployment configuration the coil is substantially circularly shaped.

26. The method of claim 25, wherein when in the deployment configuration the coil is substantially coiled within a single plane.

27. The method of claim 26, wherein the first shape conforming element and the second shape conforming element are configured so that they must be compressed in order to be substantially linearly shaped.

28. The method of claim 27, wherein the first shape conforming element and the second shape conforming element comprises a memory metal, wherein the memory metal causes the first shape conforming element and the second shape conforming element to be substantially circularly shaped when the first shape conforming element and the second shape conforming element are not compressed, and wherein the first shape conforming element and the second shape conforming element are compressed when inside a lumen of a delivery catheter.

29. The method of claim 24, the flow reducing implant comprises a radiopaque metal.

30. The method of claim 24, the flow reducing implant comprises a polymer coating configured to block blood flow through the flow diverting implant when in the deployment configuration.
